(19) United States
(12) Patent Application Publication
(10) Pub. No.: US 2024/0325149 A1
(43) Pub. Date: Oct. 3, 2024

(54) Method and System for Transcatheter Chordae Tendineae Repair

(71) Applicant: HANGZHOU VALGEN MEDTECH CO., LTD., Hangzhou (CN)

(72) Inventors: Tingchao ZHANG, Hangzhou (CN); Yang LI, Hangzhou (CN); Zhenping ZHUANG, Hangzhou (CN); Xinhuo YAN, Hangzhou, Zhejiang (CN); Haicheng LANG, Hangzhou (CN); Jianfeng ZENG, Hangzhou (CN)

(21) Appl. No.: 18/206,583

(22) Filed: Jun. 6, 2023

(30) Foreign Application Priority Data
Mar. 31, 2023 (CN) 202310378868.4

(51) Int. Cl. A61F 2/24; A61B 17/00

(52) U.S. Cl. CPC A61F 2/2457; A61B 17/00234; A61F 2/2466; A61B 2017/00243

(57) ABSTRACT

Some embodiments of the present disclosure provide a method and system for transcatheter chordae tendineae repair. The method includes: transvascular entering into an atrium of a heart; advancing a leaflet implant from an atrial side of a leaflet of the heart to a corresponding ventricular side, so as to fix the leaflet to a suture attached to the leaflet implant, where a proximal end of the suture extends out of a body to connect with a ventricular implant; delivering the ventricular implant from the atrium to a corresponding ventricle, and anchoring the ventricular implant to ventricular tissue of the corresponding ventricle; adjusting the suture to an appropriate tension; and actuating the ventricular implant to retain the suture at the ventricular implant under the appropriate tension.

### CROSS-REFERENCE TO RELATED APPLICATIONS

The present disclosure claims priority to Chinese Patent Application No. 202310378868.4, filed on Mar. 31, 2023 and entitled “Method and System for Transcatheter Chordae Tendineae Repair”, the contents of which are hereby incorporated by reference in its entirety.

### TECHNICAL FIELD

The present disclosure relates to a heart valve repair technology, and in particular to a method and system for transcatheter chordae tendineae repair.

### BACKGROUND

A mitral valve or tricuspid valve in a human heart as a one-way valve connecting an atrium and a ventricle, allows blood to flow from the atrium into the ventricle, and includes an annulus, two or three leaflets, a plurality of chordae tendineae, a papillary muscle, etc. However, when the chordae tendineae is diseased or ruptured, resulting in valve leaflet prolapse, a mitral valve or a tricuspid valve cannot be completely closed, further causing blood reflux, which calls mitral or tricuspid regurgitation.

Currently, although some systems that are less invasive than traditional thoracotomy have been proposed for insufficiency of the mitral valve or tricuspid valve caused by disease or rupture of the chordae tendineae, transcatheter repair of a heart valve will still bring additional challenges. For example, for an artificial chordae tendineae implantation, two ends of a suture must be fixed to a leaflet and a ventricular tissue respectively, so as to form artificial chordae tendineae. However, repair of the heart valve will be under the influence of a fixation mode or travel path of the suture at a leaflet end, and the number of instruments used in a single surgery. Moreover, such a transcatheter repair surgery for a patient that passes through a transcatheter system is difficult to operate and implement, further providing difficulty for the heart valve repair.

### SUMMARY

An objective of the present disclosure is to provide a method and system for transcatheter chordae tendineae repair. The method and system not only effectively guarantee a relieving regurgitation effect, but also further simplify surgery steps, thereby greatly guaranteeing a treatment effect.

In order to achieve the above objective, in a first aspect, in some embodiments of the present disclosure provide a method for transcatheter chordae tendineae repair. The method includes:
transvascular entering into an atrium of a heart;
advancing a leaflet implant from an atrial side of a leaflet of the heart to a corresponding ventricular side, so as to fix the leaflet to a suture attached to the leaflet implant, where a proximal end of the suture extends out of a body to connect with a ventricular implant;
delivering the ventricular implant from the atrium to a corresponding ventricle, and anchoring the ventricular implant to ventricular tissue of the corresponding ventricle;
adjusting the suture to an appropriate tension; and
actuating the ventricular implant to retain the suture at the ventricular implant under the appropriate tension.

In a second aspect, in some embodiments of the present disclosure further provide a system for transcatheter chordae tendineae repair. The system includes:
a delivery catheter, wherein the delivery catheter is advanced into an atrium of a heart through a vasculature of a patient, so as to provide a pathway into the heart from outside a body;
a leaflet implant to which a suture is attached, wherein the leaflet implant is configured to be advanced from an atrial side of a leaflet of the heart to a corresponding ventricular side through the delivery catheter, so as to fix the leaflet to the suture; and
a ventricular implant, wherein the suture extends proximally through the delivery catheter to be connected with the ventricular implant outside the body, and the ventricular implant is configured to be advanced from the atrium to a corresponding ventricle through the delivery catheter, so as to be anchored to ventricular tissue;
when the suture being adjusted to an appropriate tension, actuating the ventricular implant to retain the suture at the ventricular implant.

According to the method and system of the present disclosure, the leaflet implant is arranged at the leaflet to fix one end of the suture, thereby preventing a bonding edge of the leaflet from being curled and stacked due to, for example, a knotted suture mode without the leaflet implant. Moreover, the ventricular implant is implanted into the ventricular tissue (such as a papillary muscle of a left ventricle, a left ventricular free wall, a papillary muscle of a right ventricle or a right ventricular free wall), so as to tightly attach the leaflet implant to the ventricular side of the leaflet under a great pressure difference between the atrium and the ventricle, thereby avoiding blood regurgitated from a junction of the suture and the leaflet, and further effectively eliminating regurgitation at the leaflet. In addition, the present disclosure further utilizes the ventricular implant to lock the other end of the suture and anchors the ventricular implant to the ventricular tissue, thereby simplifying surgery steps, and greatly guaranteeing a treatment effect.

### BRIEF DESCRIPTION OF THE DRAWINGS

In order to describe technical solutions of embodiments of the present disclosure more clearly, accompanying drawings required for the embodiments are briefly introduced below. Apparently, the accompanying drawings in the following description are some embodiments of the present disclosure, and those of ordinary skill in the art can further derive other accompanying drawings from these accompanying drawings without making creative efforts.

FIGS. 1 and 2 illustrate schematic diagrams of an entry approach of a system for transcatheter chordae tendineae repair to enter into a mitral valve for chordae tendineae repair in some embodiments.

FIG. 3 illustrates a schematic diagram of fixing a suture to a leaflet via a pathway entering a mitral valve.

FIGS. 4a-6b illustrate schematic diagrams of implanting different leaflet implants into a leaflet by utilizing a puncture assembly.

FIGS. 7a-7c illustrate a schematic diagram of fixing a short suture to a leaflet by utilizing a puncture assembly.

FIG. 8 illustrates a schematic diagram of capturing a leaflet by utilizing a capture assembly.

FIGS. 9-15 illustrate utilization of different capture assemblies in cooperation with a puncture assembly to complete fixation of a suture to a leaflet.

FIGS. 16 and 17 illustrate a ventricular implant entering a ventricle via a pathway entering a mitral valve.

FIGS. 18 and 19 illustrate schematic diagrams of inserting a locator into ventricular tissue, so as to stabilize a ventricular implant.

FIGS. 20 and 21 illustrate schematic diagrams of anchoring a ventricular implant into ventricular tissue.

FIGS. 22 and 23 illustrate schematic diagrams of adjusting a suture to an appropriate tension.

FIG. 24 illustrates a schematic enlarged diagram of a portion V shown in FIG. 23.

FIGS. 25-27 illustrate different ventricular implants.

FIG. 28 illustrates a schematic diagram of completing anchoring of a ventricular implant in some embodiments.

FIG. 29 illustrates a schematic diagram of introducing a suture cutter in some embodiments shown in FIG. 28.

FIG. 30 illustrates a schematic diagram of completing implantation of artificial chordae tendineae in some embodiments.

FIG. 31 illustrates a schematic diagram of completing anchoring of a ventricular implant in other embodiments.

FIG. 32 illustrates a schematic diagram of withdrawing a traction member in other embodiments shown in FIG. 31.

FIG. 33 illustrates a schematic diagram of completing implantation of artificial chordae tendineae in other embodiments.

The following detailed description will further describe the present disclosure with reference to the accompanying drawings described above.

### DETAILED DESCRIPTION OF THE EMBODIMENTS

The technical solutions in the embodiments of the present disclosure are clearly and completely described below in combination with the accompanying drawings in the embodiments of the present disclosure. Apparently, the examples described are only some examples rather than all examples of the present invention. On the basis of the examples of the present invention, all other examples obtained by those of ordinary skill in the art without making inventive efforts all fall within the scope of protection of the present invention.

In addition, the description of the following embodiments refers to additional illustrations to illustrate specific embodiments that the present disclosure can implement. Directional terms mentioned in the present disclosure, such as “upper”, “lower”, “front”, “back”, “left”, “right”, “inside”, “outside”, “side surface”, etc., only refer to the direction shown in the accompanying drawings. Therefore, the directional terms used are for better and clearer description and understanding of the present disclosure, rather than indicating or implying that a device or element referred to must have a particular orientation, be constructed and operated in a specific orientation, and therefore will not be understood as a limitation of the present disclosure.

It should be noted that in order to more clearly describe a method and system for transcatheter chordae tendineae repair according to the present disclosure, the limiting terms “proximal end” and “distal end” mentioned in the description of the present disclosure are both customary terms in the field of interventional medicine. Specifically, “distal end” refers to one end away from an operator during surgery operation, and “proximal end” refers to one end close to the operator during surgery operation; a direction of a rotation center axis of a type of objects such as columns and tubes is defined as an axial direction; a circumferential direction refers to a direction around an axis of the type of objects such as columns or tubes (perpendicular to the axis and a cross-sectional radius); and a radial direction is a direction along a diameter or radius. It is worth noting that “end” appearing in words of “proximal end”, “distal end”, “one end”, “the other end”, “first end”, “second end”, “initial end”, “tail end”, “two ends”, “free end”, “upper end”, and “lower end” is not limited to an end head, an end point, or an end surface, but also includes a portion of a self-end head, the end point, or the end surface at an element, to which the end head, the end point, or the end surface belongs, extending by a certain axial direction and/or radial distance. Unless otherwise defined, all technical and scientific terms used in the present disclosure have the same meanings as those commonly understood by those skilled in the technical field of the present disclosure. The customary terms used in the description of the present disclosure are only for the purpose of describing specific embodiments, and cannot be understood as limitations to the present disclosure.

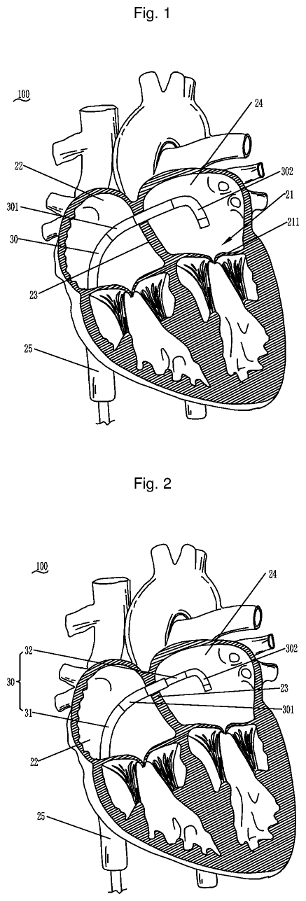

Some embodiments of the present disclosure provide a method and system for transcatheter chordae tendineae repair. The method and system can obtain a pathway entering into a heart valve through an intravascular, transcatheter approach. Certainly, if the heart valve being repaired is a mitral valve, the heart valve can further be accessed via transseptal approach. FIGS. 1 and 2 show schematic diagrams of a system for transcatheter chordae tendineae repair 100 entering into a mitral valve 21 for tendineae repair in some embodiments. The system for transcatheter chordae tendineae repair 100 includes a delivery catheter 30. The delivery catheter 30 provides a pathway entering into a heart from an exterior of a patient's body. The delivery catheter 30 enters a right atrium 22 of the heart through the vasculature of a patient, and further enters a left atrium 24 through an atrial septum 23. An opening at a distal end of the delivery catheter 30 is located in the left atrium 24, and other instruments or components enter the heart by means of the pathway provided by the delivery catheter 30 for completing tendineae repair of the mitral valve 21, so as to prevent mitral regurgitation.

In some embodiments, as shown in FIG. 1, the delivery catheter 30 is an elongated flexible catheter. The distal end of the delivery catheter 30 is provided with a first bending section 301 and a second bending section 302, so as to achieve at least two stages of bending function. In the embodiment, the second bending section 302 is disposed at a location of the delivery catheter 30 close to the opening at the distal end of the delivery catheter 30, and the first bending section 301 is disposed beside a proximal end of the second bending section 302. After a puncture tool is used to puncture the atrial septum 23, the delivery catheter 30 passes through the vasculature of the patient such as inferior vena cava 25, and enters into the left atrium 24 from the right atrium 22 through the atrial septum 23, so that the opening at the distal end of the delivery catheter 30 is located in the left atrium 24. In this case, the first bending section 301 is located in the right atrium 22 to adjust a first direction and a first angle of the delivery catheter 30 near a location of a puncture point of the atrial septum 23, so as to adjust the delivery catheter 30 from approximately parallel to the atrial septum 23 to approximately perpendicular to the atrial septum 23, so as to make the delivery catheter 30 pass vertically through the puncture point of the atrial septum 23. The second bending section 302 is located in the left atrium 24, and is configured to match the first bending section 301 to adjust a second direction and a second angle of the delivery catheter 30, so as to reposition the delivery catheter 30 from approximately perpendicular to the atrial septum 23 to approximately parallel to the atrial septum 23, so as to make the distal end of the delivery catheter 30 approximately perpendicular to an annulus of the mitral valve 21, thereby providing a desired pathway for other instruments or components to repair the heart valve.

In other embodiments, as shown in FIG. 2, the delivery catheter 30 includes an outer delivery catheter 31 and an inner delivery catheter 32. A distal end of the outer delivery catheter 31 is provided with a first bending section 301, and a distal end of the inner delivery catheter 32 is provided with a second bending section 302. After the puncture tool is used to puncture the atrial septum 23, the outer delivery catheter 31 passes through the vasculature of a patient such as an inferior vena cava 25, and enters into the right atrium 22. Further, the first bending section 301 of the outer delivery catheter 31 is further used to adjust the outer delivery catheter 31 from approximately parallel to the atrial septum 23 to approximately perpendicular to the atrial septum 23. Then, the outer delivery catheter 31 passes vertically through the puncture point of the atrial septum 23 and enters into the left atrium 24, so that the distal end of the outer delivery catheter 31 is located in the left atrium 24. The inner delivery catheter 32 can be axially advanced along a lumen of the outer delivery catheter 31 and enter into the left atrium 24. In some embodiments, the distal end of the inner delivery catheter 32 extends out of an opening at the distal end of the outer delivery catheter 31. The inner delivery catheter 32 is adjusted from approximately perpendicular to the atrial septum 23 to approximately parallel to the atrial septum 23 by the second bending section 302 of the inner delivery catheter 32 extending out the opening at the distal end of the outer delivery catheter, so as to make the distal end of the inner delivery catheter 32 approximately perpendicular to the annulus of the mitral valve 21, thereby providing a desired pathway for other instruments or components to repair the heart valve.

In some embodiments, the opening at the distal end of the delivery catheter 30 is located approximately in a middle of the annulus of the mitral valve 21, so as to avoid the risk of regurgitation caused by compression to a leaflet 211 of the mitral valve 21 formed by other instruments or components entering the mitral valve 21 subsequently via the delivery catheter 30.

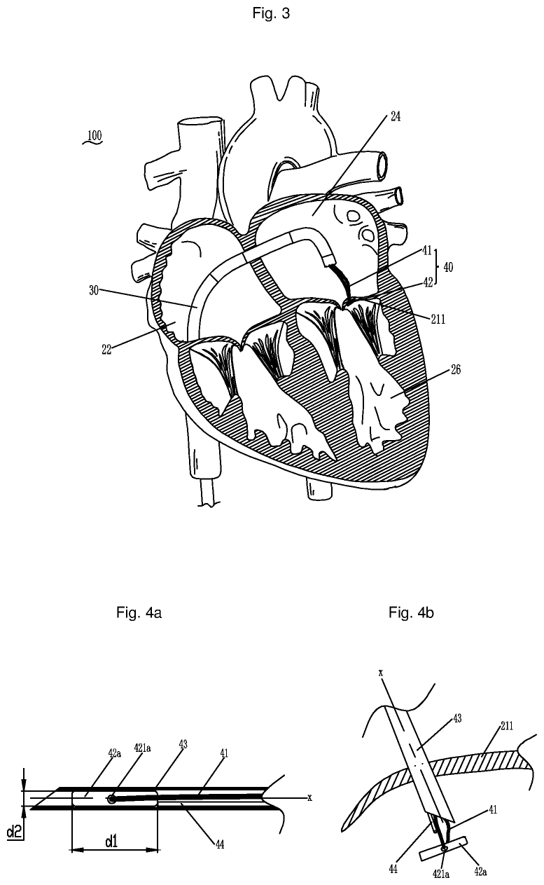

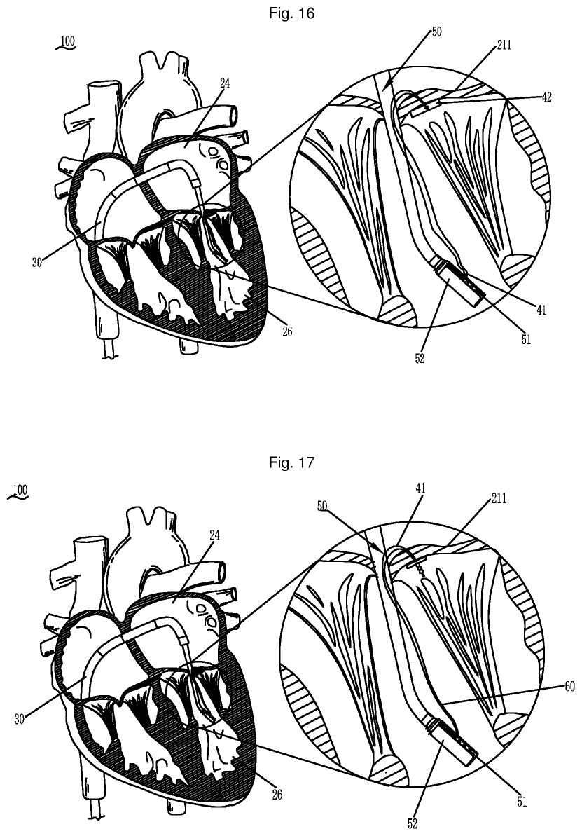

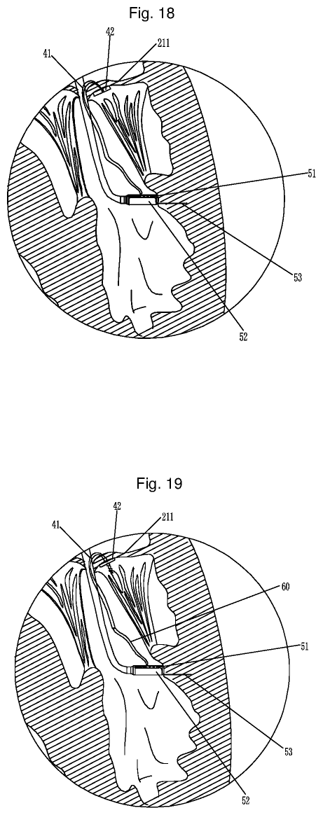

FIGS. 3-33 show schematic diagrams of various steps of a method for transcatheter chordae tendineae repair in some embodiments. As shown in FIG. 3, a system for transcatheter chordae tendineae repair 100 further includes a leaflet implantation subsystem 40. The leaflet implantation subsystem 40 includes a suture 41 and a leaflet implant 42 attached to one end of the suture 41. After the delivery catheter 30 is advanced into the heart such as the left atrium 24, the leaflet implant 42 to which the suture 41 is attached, is delivered via the delivery catheter 30 to a target location of the leaflet 211, and is advanced to a corresponding ventricular side from an atrial side of the leaflet 211 of the mitral valve 21, so as to fix the suture 41 at the leaflet 211. In this case, a distal end of the suture 41 is attached to the leaflet implant 42, the leaflet 211 is fixed to the suture 41, and a proximal end of the suture 41 is pulled to extend out of the patient's body along the delivery catheter 30. In some embodiments, in order to guarantee a connecting force between the suture 41 and the leaflet 211, a fixed location of the suture 41 on the leaflet 211, which is the target location of the leaflet 211 to which the leaflet implant 42 is delivered, needs to guarantee a certain distance from a bonding edge of the leaflet 211. In the embodiment, the leaflet implant 42 is advanced onto the leaflet 211 in a range of 5 mm to 8 mm from the bonding edge of the leaflet 211, so as to achieve fixation to the leaflet 211.

Certainly, in other embodiments, when the heart valve being repaired is a tricuspid valve, after the delivery catheter 30 is advanced into the right atrium 22 of the heart, the leaflet implant 42 to which the suture 41 is attached, is delivered via the delivery catheter 30 to a target location of the right atrium 22, and the same method described above is used to fix a leaflet of the tricuspid valve to the suture 41.

In view of a fixation mode without the leaflet implant in the art known to inventors, for example, a knotted suture mode is likely to cause the bonding edge of the leaflet to curl and stack, thereby reducing an effective depth of the bonding edge, and further, there is a risk of a poor regurgitation repair effect or even incapability to repair regurgitation. Compared to the fixation mode without the leaflet implant in the art known to inventors, another fixation mode in the present disclosure by disposing the leaflet implant at the leaflet, thereby preventing the bonding edge of the leaflet from being curled and stacked, and further effectively reducing a degree of regurgitation of the leaflet. In addition, according to the present disclosure, the leaflet implant is implanted into the ventricular side of the leaflet (such as a left ventricular side of the leaflet of the mitral valve or a right ventricular side of the leaflet of the tricuspid valve). During contraction of the heart, since a pressure in the ventricular is much greater than a pressure in the atrial, the leaflet implant is tightly attached to the ventricle side of the leaflet under a huge pressure difference, thereby avoiding regurgitation of blood from a junction between the suture and the leaflet, and further guaranteeing a treatment effect.

Certainly, in other embodiments, the leaflet implant to which the suture is attached can also advanced from the ventricular side of the leaflet (such as the left ventricular side or the right ventricular side) to the corresponding atrial side of the leaflet (such as a left atrial side of the leaflet of the mitral valve or a right atrial side of the leaflet of the tricuspid valve), so that the leaflet is fixed to the suture, so as to fix the suture to the leaflet.

With reference to FIGS. 4a-7c, further, the leaflet implant 42 to which the suture 41 is attached is delivered by a puncture assembly 43. The puncture assembly 43 is advanced along an axial direction of the delivery catheter 30 and punctures the leaflet 211 from an atrium, such as the left atrium 24, to a corresponding ventricle, such as the left ventricle 26, and releases the leaflet implant 42 loaded in the puncture assembly 43, such that the leaflet implant 42 is advanced from the atrial side of the leaflet 211 to the ventricular side of the leaflet 211. The puncture assembly 43 is withdrawn, the leaflet implant 42 can be left to abut against the ventricular side of the leaflet 211, such that the suture 41 is fixed to the leaflet 211.

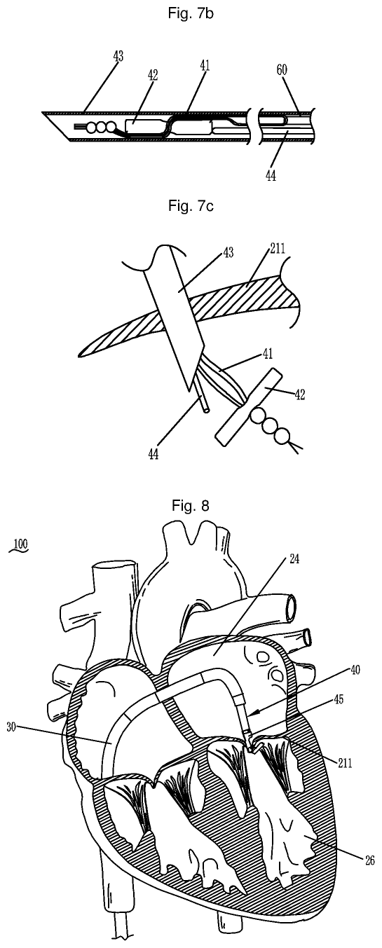

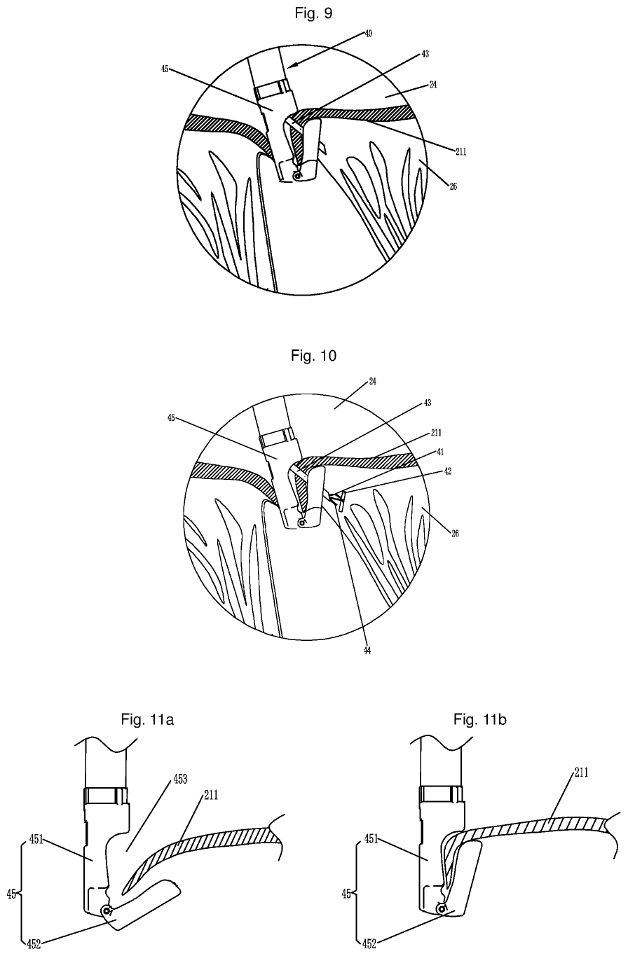

Certainly, the leaflet implant 42 to which the suture 41 is attached can be advanced towards a distal end of the puncture assembly 43 by a push rod 44 accommodated in the puncture assembly 43, so as to pass out of the puncture assembly 43 and be releases to the ventricular side of the leaflet 211. The puncture assembly 43 has at least one cavity channel for accommodating the leaflet implant 42 and the push rod 44, the cavity channel can penetrate the puncture assembly 43 and extend from the distal end of the puncture assembly 43 to a proximal end of the puncture assembly 43. In some embodiments, the leaflet implant 42 is preloaded at a distal end of the cavity channel of the puncture assembly 43, and the push rod 44 is loaded at the proximal end of the leaflet implant 42. The distal end of the suture 41 is attached to the leaflet implant 42, and the proximal end of the suture 41 passes through the puncture assembly 43 to extend out of the patient's body along the delivery catheter 30. The proximal end of the suture 41 can extend directly out of the body (see FIGS. 4a-6b), or can be pulled by means of a traction member 60 (see FIGS. 7a-7c) to indirectly extend out of the body. It could be understood that the puncture assembly 43 is advanced to the target location (which is also referred to as a puncture point) of the leaflet 211 in a direction approximately perpendicular to the leaflet 211 and punctures the leaflet 211 from the atrial side of the leaflet 211 to the corresponding ventricular side, so as to completely expose an opening at the distal end of the puncture assembly 43 at the ventricular side. Next, the push rod 44 is axially advanced towards the distal end of the puncture assembly 43, so as to push the leaflet implant 42 out of the distal end of the puncture assembly 43 for releasing the leaflet implant 42.

In some embodiments, the leaflet implant 42 is rod-shaped. As shown in FIGS. 4a and 4b, a rod-shaped leaflet implant 42a is provided with at least one through hole configured to fix a suture 41. As shown in FIG. 4a, one first through hole 421a is provided on the rod-shaped leaflet implant 42a, and the first through hole 421a passes through a central region of the rod-shaped leaflet implant 42a in a radial direction of the rod-shaped leaflet implant 42a. The suture 41 passes through the first through hole 421a and is folded in half to form two free ends. The rod-shaped leaflet implant 42a to which the suture 41 is attached is preloaded in the puncture assembly 43, in this case, an axis of the rod-shaped leaflet implant 42a is substantially parallel to or overlapping with a central axis X of the puncture assembly 43, and the two free ends of the suture 41 extend towards the proximal end of the puncture assembly 43 to pass through the puncture assembly 43 out of the patient's body. As shown in FIG. 4b, a push rod 44 is advanced axially towards the distal end of the puncture assembly 43, so as to completely push the rod-shaped leaflet implant 42a out of the opening at the distal end of the puncture assembly 43. After the rod-shaped leaflet implant 42a is completely released, the rod-shaped leaflet implant 42a rotates under the action of gravity, so as to be changed from a folded state parallel to the puncture assembly 43 to an unfolded state with an angle to the puncture assembly 43. That is, when the rod-shaped leaflet implant 42a is in the unfolded state, the angle is formed between the axis of the rod-shaped leaflet implant 42a and the central axis X of the puncture assembly 43. In some embodiments, the angle between the axis of the rod-shaped leaflet implant 42a and the central axis X of the puncture assembly 43 is 90 degrees. In view that an axial dimension d1 of the rod-shaped leaflet implant 42a is much greater than a radial dimension d2 of the rod-shaped leaflet implant 42a, after the rod-shaped leaflet implant 42a rotates, the axial dimension d1 of the rod-shaped leaflet implant 42a is necessarily greater than a diameter of a puncture hole in which the puncture assembly 43 punctures the leaflet 211, and then after the puncture assembly 43 is withdrawn, the rod-shaped leaflet implant 42a is necessarily left at the ventricular side of the leaflet 211. In particular, it should be explained that in some embodiments, a range of the axial dimension d1 of the rod-shaped leaflet implant 42a is 5 mm to 10 mm. Certainly, the range of the axial dimension d1 can also be freely set according to actual requirements of a user and other considerations. For example, the axial dimension d1 should be selected to avoid at least one of considerations such as the rod-shaped leaflet implant 42a falling off from the puncture hole, the leaflet 211 being curled after the rod-shaped leaflet implant 42a is implanted, and the rod-shaped leaflet implant 42a causing damage to other tissue.

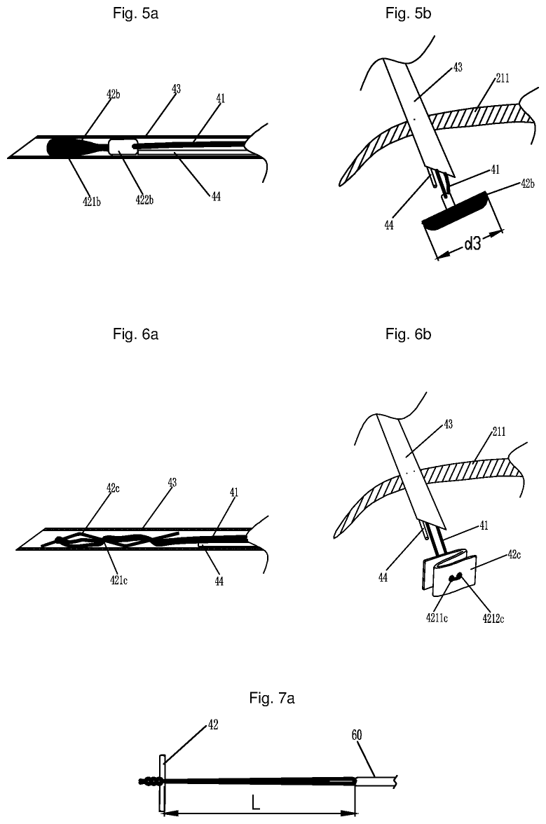

In other embodiments, the leaflet implant 42 is an expandable implant. As shown in FIGS. 5a and 5b, the expandable implant is an expandable disc 42b. The expandable disc 42b includes a braided mesh disc 421b and at least one connection sleeve 422b. The braided mesh disc 421b has expandable and shape memory characteristics, and for example, the braided mesh disc 421b is braided by a nickel-titanium wire. The connection sleeve 422b is fixedly disposed at a proximal end of the braided mesh disc 421b. The distal end of the suture 41 is connected to the proximal end of the connection sleeve 422b. For example, the proximal end of the connection sleeve 422b has a through hole (not shown), the distal end of the suture 41 can pass through the through hole of the connection sleeve 422b to connect. As shown in FIG. 5a, the braided mesh disc 421b is preloaded in the puncture assembly 43, at least a radial dimension of the braided mesh disc 421b is limited to be accommodated in the distal end of the puncture assembly 43, and the other end of the suture 41 extends towards the proximal end of the puncture assembly 43 to pass through the puncture assembly 43 out of the patient's body. As shown in FIG. 5b, a push rod 44 is axially advanced towards the distal end of the puncture assembly 43, so as to completely push the expandable disc 42b out of the opening at the distal end of the puncture assembly 43. Then, the expandable disc 42b is released from the distal end of the puncture assembly 43, and due to the expandable and shape memory characteristics of the braided mesh disc 421b, the braided mesh disc 421b returns to an original shape. For example, a part or whole of the radial dimension of the braided mesh disc 421b is increased. In this case, a radial dimension d3 of the braided mesh disc 421b after being expanded is necessarily greater than the diameter of the puncture hole at which the puncture assembly 43 punctures the leaflet 211. After the puncture assembly 43 is withdrawn, the expandable disc 42b is necessarily left on the ventricular side of the leaflet 211. Certainly, the radial dimension d3 of the braided mesh disc 421b after being expanded can further be configured to be not less than an outer diameter of the puncture assembly 43. In some embodiments, a range of the radial dimension d3 is 5 mm to 10 mm.

Certainly, in other embodiments, the leaflet implant 42 is flexible, and for example, the leaflet implant 42 is a flexible gasket 42c having a variable radial cross section. As shown in FIGS. 6a and 6b, the flexible gasket 42c is an elongated strip and can be axially folded into at least one fold, the flexible gasket 42c after being axially folded includes a plurality of flexible gasket sections, and at least one second through hole 421c is provided on each of the plurality of flexible gasket sections. In some embodiments, the flexible gasket 42c can be axially folded into three folds, and two second through holes 421c disposed at intervals are provided on each of the plurality of flexible gasket sections. The two second through holes 421c are respectively a first flexible gasket through hole 4211c and a second flexible gasket through hole 4212c, one end of the suture 41 enters and penetrates each first flexible gasket through hole 4211c (or each second flexible gasket through hole 4212c) from a proximal end of the flexible gasket 42c, and passes out from a distal end of the flexible gasket 42c, then further enters and penetrates each second flexible gasket through hole 4212c (or each first flexible gasket through hole 4211c) from the distal end of the flexible gasket 42c, and passes out from the proximal end of the flexible gasket 42c, and then is folded in half to form two free ends. As shown in FIG. 6a, the flexible gasket 42c is preloaded in the puncture assembly 43, and a gap is provided between adjacent two flexible gasket sections of the flexible gasket 42c. As shown in FIG. 6b, a push rod 44 is axially advanced towards the distal end of the puncture assembly 43, so as to completely push the flexible gasket 42c out of the opening at the distal end of the puncture assembly 43. Further, under a tensioning effect of the suture 41, the flexible gasket 42c can be tightened, folded and axially contracted, thereby forming a radially enlarged and axially shortened structure, so as to form a sufficient radial cross section. The radial cross section can avoid the risk of the flexible gasket 42c falling out of the puncture hole of the leaflet 211 after being pulled. In particular, it should be explained that all first flexible gasket through holes 4211c are retained coaxial after the flexible gasket 42c being folded. All the second flexible gasket through holes 4212c are retained coaxial after the flexible gasket 42c being folded, so as to avoid an uncontrollable risk caused by winding of the suture 41 at the flexible gasket 42c.

It could be understood that the proximal end of the suture 41 can directly pass out of the patient's body in a manner described in FIGS. 4a-6b, and an operator can directly correspondingly operate the proximal end of the suture 41 outside the body. Further, the proximal end of the suture 41 can indirectly pass out of the body in a mode as shown in FIGS. 7a-7c. For example, the suture 41 extends out of the body by pulling a traction member 60. In this case, the proximal end of the suture 41 is located inside the heart, the traction member 60 is connected with the proximal end of the suture 41. The traction member 60 extends proximally out of the patient's body, and the operator can pull the traction member 60 outside the body to indirectly operate the suture 41.

With reference to FIG. 7a, the distal end of the suture 41 is attached to the leaflet implant 42, and the proximal end of the suture 41 is detachably connected with a distal end of the traction member 60. In the embodiment, after the suture 41 is folded in half, two free ends of the suture are embedded onto the leaflet implant 42 with a knot, and a loop is formed at the proximal end of the suture 41. After the traction member 60 passes through the loop of the suture 41, the traction member 60 is folded in half towards the proximal end to detachably connect with the suture 41. Certainly, in other embodiments, the traction member 60 can further be detachably connected with the suture 41 by means of a thread or a buckle. In the embodiment, since an effective length L of the suture 41 attached to the leaflet implant 42 is short (the effective length L is an axial length from the proximal end of the suture 41 to the leaflet implant 42), a total length of the suture 41 for single use is greatly shortened compared to the art known to inventors, thereby greatly reducing material cost. Certainly, in order to reduce an effect of the suture 41 on a repair effect of the chordae tendineae during a surgery, in some embodiments, a range of the effective length L of the suture 41 is 10 mm to 50 mm. In some embodiments, a range of a diameter of the suture 41 is between 0.3 mm and 0.6 mm, and a material of the suture 41 is polytetrafluorethylene (ePTFE). It could be understood that the effective length L of the suture 41 can further be designed into a plurality of specifications, so as to be adapted to different requirements of different patients for the length of the suture 41. Therefore, a doctor can confirm the most appropriate length of an artificial chordae tendineae by means of a medical image before the surgery, and select the suture 41 having a corresponding specification, such that complicated operation and suture contamination when the suture 41 having an excessively long length extends towards the proximal end in a delivery catheter 30 can be avoided, and the redundant suture 41 can also be prevented from being cut, thereby saving surgery steps, and simplifying an instrument.

Next, the leaflet implant 42 and the traction member 60 shown in FIG. 7a are integrally preloaded in the puncture assembly 43. As shown in FIGS. 7b and 7c, the leaflet implant 42 is accommodated at the distal end of the puncture assembly 43, and the proximal end of a suture 41 is brought by the traction member 60 to pass through the proximal end of the puncture assembly 43 and extend out of the patient's body. A push rod 44 is axially advanced toward the distal end of the puncture assembly 43, the leaflet implant 42 is completely pushed out of the opening at the distal end of the puncture assembly 43, and rotates under the action of gravity, to be changed from the folded state to the unfolded state. The folded state and the unfolded state shown in FIGS. 7a-7c are the same as those shown in FIGS. 4a and 4b, which will not be repeated herein. Finally, the puncture assembly 43 is withdrawn, the leaflet implant 42 is left at the ventricle side of the leaflet 211, the proximal end of the suture 41 is also left inside the heart, and the proximal end of the suture 41 extends out of the body along the delivery catheter 30 by the traction member 60, so as to adjust the suture 41 by the operator, and further introduce an instrument along a pathway of the suture 41.

In some embodiments, the traction member 60 can be a traction wire, such as a stainless steel wire, a nickel-titanium wire or a tungsten wire. The traction member 60 can also be a traction rope, such as a stainless steel wire rope, a tungsten wire rope or a nickel-titanium wire rope. The traction member 60 can further be a polymer braided wire, such as a polyethylene terephthalate (PET) wire or an ultra-high molecular weight polyethylene wire.

In some embodiments, in order to prevent the leaflet 211 from affecting a puncture effect of the puncture assembly 43 due to beating of the heart, before the puncture assembly 43 implements puncture, a capture assembly captures the leaflet 211, so as to limit movement of the leaflet 211. As shown in FIGS. 8-10, the leaflet implantation subsystem 40 further includes a capture assembly 45, the capture assembly 45 can be delivered via the delivery catheter 30 until the distal end of the capture assembly 45 is beyond the distal end of the delivery catheter 30. Certainly, in other embodiments, the capture assembly 45 can also be delivered together with the delivery catheter 30. In some embodiments, as shown in FIG. 8, the capture assembly 45 is pushed from the delivery catheter 30 to the atrium, and further travels in a direction approximately perpendicular to the annulus to reach the bonding edge of the leaflet 211. It could be understood that in some embodiments, the capture assembly 45 and the puncture assembly 43 can be simultaneously and parallelly loaded in the delivery catheter 30. After the capture assembly 45 extends out of the delivery catheter 30 and captures the leaflet 211, the puncture assembly 43 extends out of the delivery catheter 30, performs puncture at the puncture point of the leaflet 211, and pushes the leaflet implant 42 out, so as to fix one end of the suture 41 to the leaflet 211. In other embodiments, as shown in FIGS. 9 and 10, the capture assembly 45 is provided with at least one hollow channel (not shown) extending axially, and the puncture assembly 43 is preloaded in the hollow channel of the capture assembly 45 and extends proximally. After the capture assembly 45 extends out of the distal end of the delivery catheter 30 and captures the leaflet 211, the puncture assembly 43 then extends out of an distal end of the hollow channel of the capture assembly 43 and further performs puncture from an atrial side of the leaflet 211 to the corresponding ventricle side, so as to completely expose an opening at a distal end of the puncture assembly 43 at the ventricular side. The push rod 44 is axially advanced towards the distal end of the puncture assembly 43, and the leaflet implant 42 to which the suture 41 is attached extends out of the opening at the distal end of the puncture assembly 43 and is released, so as to abut against the ventricular side. Apart from the leaflet implant 42 to which the suture 41 is attached and the traction member 60 left, other components of the leaflet implantation subsystem 40 are all withdrawn from the heart, such that a subsequent instrument can enter into the heart from the pathway provided by the delivery catheter 30 and complete the subsequent operation. A state after withdrawn refers to FIG. 3.

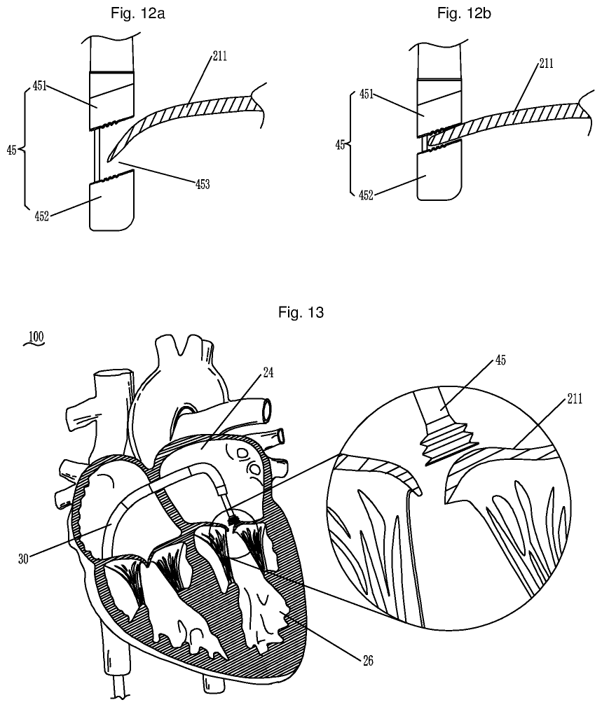

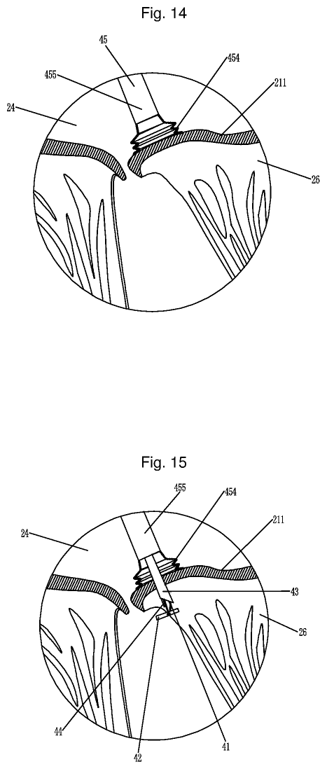

It could be understood that the capture assembly 45 can capture the leaflet 211 in a clamping manner or a suction manner, so as to provide an effective guarantee for the puncture assembly 43 to better achieve puncture. The effective guarantee includes, but not limited to, guaranteeing stability of puncture, controllability of a puncture depth, stability of a puncture force, etc. As shown in FIGS. 11a-12b, the capture assembly 45 includes a first clamping arm 451 and a second clamping arm 452 movable relative to the first clamping arm 451. When the capture assembly 45 is in an open state (see FIG. 11a or FIG. 12a), a clamping region 453 is formed between the second clamping arm 452 and the first clamping arm 451. After the leaflet 211 enters into the clamping region 453, the second clamping arm 452 is driven to move to clamp the leaflet 211 between the second clamping arm 452 and the first clamping arm 451, and then the capture assembly 45 is being changed to a closed state (see FIG. 11b or FIG. 12b). In some embodiments, as shown in FIGS. 11a and 11b, the second clamping arm 452 can rotate relative to the first clamping arm 451, and a controllable angle is formed between the second clamping arm 452 and the first clamping arm 451, so as to provide the clamping region 453 for the leaflet 211 and to clamp the leaflet 211. In other embodiments, as shown in FIGS. 12a and 12b, the second clamping arm 452 can move axially relative to the first clamping arm 451, and a controllable distance is formed between the second clamping arm 452 and the first clamping arm 451, so as to provide the clamping region 453 for the leaflet 211 and to clamp the leaflet 211.

In some embodiments, as shown in FIGS. 13-15, the capture assembly 45 is a suction cup structure having a suction function, and includes a suction cup 454 disposed at the distal end of the capture assembly 45 and a hollow sealing lumen 455 extending proximally from a distal end of the suction cup 454. When the suction cup 454 is attached to the target location of the leaflet 211, such as the puncture point, a proximal end of the capture assembly 45 is driven to apply a certain suction force to draw out containments in the sealing lumen 455. In this case, the sealing lumen 455 forms a negative pressure, and sucks the leaflet 211, so as to achieve a capture function. In the embodiment, the puncture assembly 43 is preloaded in the sealing lumen 455. After the suction cup 454 completes capturing of the leaflet 211, the puncture assembly 43 performs puncture, so as to complete fixation of the suture 41 at the leaflet 211.

It should be explained that the puncture assembly 43, the push rod 44, the capture assembly 45, etc. can be driven by a actuator or a handle connected with the corresponding proximal end of the puncture assembly 43, the push rod 44, the capture assembly 45, respectively, so as to achieve functions of puncture, release and capture.

Once the leaflet implant 42 is configured to the leaflet 211, the suture 41 extends proximally to directly or indirectly connect with a ventricular implant 51 outside of the patient's body (see FIGS. 16 and 17). In some embodiments, the proximal end (i.e., free end) of the suture 41 passes through a proximal end of the delivery catheter 30 to the outside of the body, and the operator connects the suture 41 to the ventricular implant 51 outside of the body. For example, the suture 41 passes through the ventricular implant 51. In other embodiments, the proximal end (i.e., free end) of the suture 41 is connected to the distal end of the traction member 60, the suture 41 is left inside the heart, the traction member 60 passes through the delivery catheter 30 to extend to outside of the body, and the operator connects the traction member 60 to the ventricular implant 51 outside the body to indirectly connect the suture 41 with the ventricular implant 51.

In some embodiments, with reference to FIGS. 16-21, the system for transcatheter chordae tendineae repair 100 further includes a ventricular implantation subsystem 50, and the ventricular implantation subsystem 50 includes the ventricular implant 51. Once the suture 41 and the ventricular implant 51 are connected, the ventricular implantation subsystem 50 delivers the ventricular implant 51 from the atrium into the corresponding ventricle via the delivery catheter 30, and anchors the ventricular implant 51 to ventricular tissue of the corresponding ventricle. The ventricular tissue includes, but not limited to, a papillary muscle, a free wall, etc.

It could be understood that the ventricular implantation subsystem 50 further includes an anchor catheter 52, wherein the ventricular implant 51 is delivered by the anchor catheter 52. The ventricular implant 51 is preloaded at a distal end of the anchor catheter 52. The anchor catheter 52 is advanced into the heart, such as the atrium, through the delivery catheter 30, and then further penetrates from the atrium of the leaflet 211 to the corresponding ventricle, such that an opening at the distal end of the anchor catheter 52 is attached to a target anchor region of the ventricular tissue. Then, the ventricular implant 51 loaded at the distal end of the anchor catheter 52 can be driven by an anchor actuator (not shown) detachably connected to a proximal end of the ventricular implant 51, so as to helically or axially advance the ventricular implant 51 to be anchored to the ventricular tissue. In some embodiments, the ventricular implantation subsystem 50 further includes a locator 53, and the locator 53 extends axially to pass through an anchor portion 511 of the ventricular implant 51 or extends axially beside the anchor portion 511 in the anchor catheter 52. The locator 53 is inserted into the ventricular tissue to stabilize the ventricular implant 51, and the ventricular implant 51 is then anchored into the ventricular tissue to achieve anchoring.

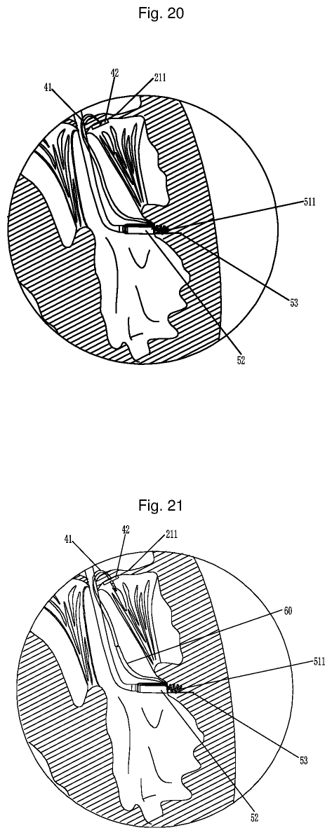

As shown in FIGS. 20 and 21, the ventricular implant 51 includes the anchor portion 511, wherein the anchor portion 51 is helically or axially advanced towards ventricular tissue by the anchor actuator, so as to anchor the anchor portion 511 to the ventricular tissue. The anchor portion 511 can be inserted along a direction approximately perpendicular to an inner surface of the ventricular tissue. Certainly, due to different locations of the ventricular tissue, the anchor portion 511 can also be inserted at an inserting angle with the inner surface of the ventricular tissue. For example, a range of the inserting angle is between 45° and 90°. In addition, a range of an anchoring depth of the anchor portion 511 anchored to the ventricular tissue is between 5 mm and 10 mm, and the anchoring depth represents an effective length of the anchor portion 511 combined with the ventricular tissue.

In some embodiments, the anchor portion 511 is a helical coil, the locator 53 is an elongated locating needle having a sharp distal end, and the locator 53 and the anchor portion 511 are parallelly loaded at the distal end of the anchor catheter 52. The locator 53 is driven by a locator driver (not shown) to extend out of the opening at the distal end of the anchor catheter 52 to insert into the ventricular tissue, such that the anchor portion 511 is prevented from being displaced during anchoring. That is, the locator 53 can retain a location of the anchor portion 511 against a rotational force transmitted from the anchor catheter 52, so as to guarantee stability when the anchor portion 511 is implanted. After the inserting of the locator 53 is completed, the anchor portion 511 is rotatably driven by the anchor actuator in the anchor catheter 52 to anchor the anchor portion 511 into the ventricular tissue.

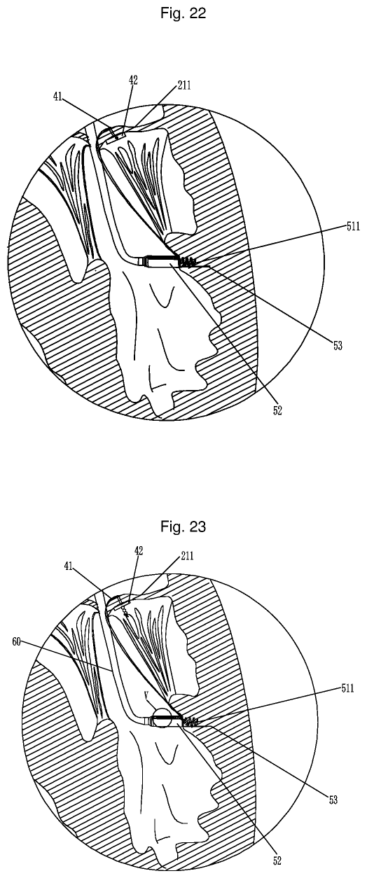

With reference to FIGS. 22 and 23, once the ventricular implant 51 is anchored to ventricular tissue, the suture 41 is adjusted to an appropriate tension, so as to achieve a best valve function. One or more groups of sutures 41 can be implanted, adjusting the tension of all the sutures 41 newly implanted until the best valve function is achieved.

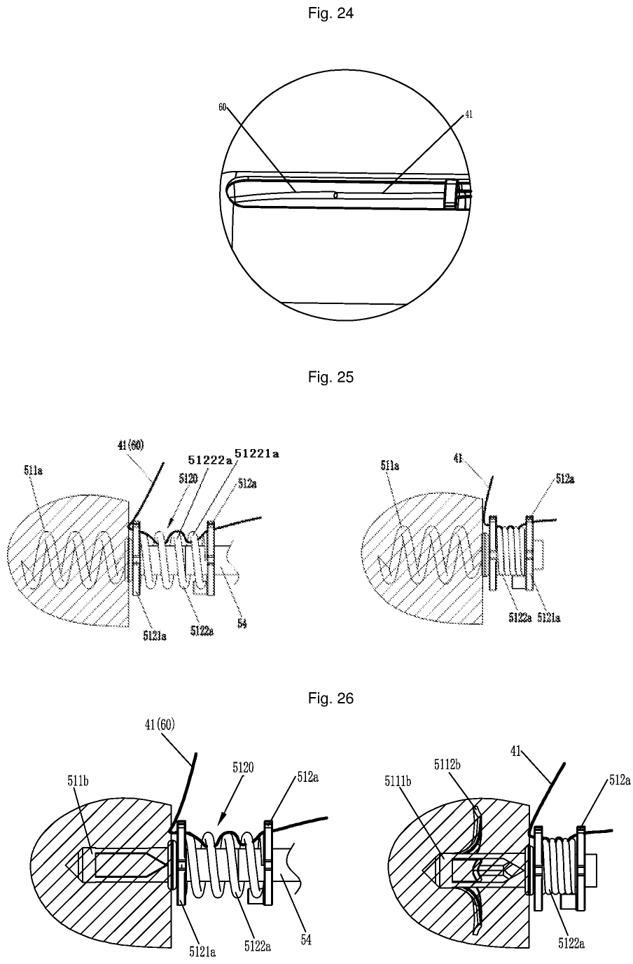

In some embodiments, as shown in FIG. 22, a length of the suture 41 located between the leaflet 211 and the ventricular implant 51 can be adjusted by pulling the proximal end of the suture 41 outside the body to adjust the tension of the suture 41 to achieve the best valve function. In other embodiments, as shown in FIG. 23, the proximal end of the suture 41 is connected to the distal end of the traction member 60. The traction member 60 extends out of the patient's body to connect with the ventricular implant 51 outside the body, and the tension of the suture 41 is adjusted by pulling the traction member 60 outside the body. In the embodiments, before the tension of the suture 41 is adjusted, the ventricular implant 51 retains to be connected with the traction member 60. The proximal end of the suture 41, i.e., a connecting end between the suture 41 and the traction member 60, is always located between the leaflet 211 and the ventricular implant 51 (see FIGS. 17, 19 and 21). In this case, the suture 41 is not in contact with or connected to the ventricular implant 51. Once the tension of the suture 41 starts to be adjusted, for example, the traction member 60 is pulled proximally, as shown in FIGS. 23 and 24, the proximal end of the suture 41 located outside the ventricular implant 51 is brought by the traction member 60 into the ventricular implant 51, so as to directly connect the suture 41 with the ventricular implant 51. At this moment, at least partially of the proximal end of the suture 41 are connected with the ventricular implant 51.

As shown in FIGS. 25-27, the ventricular implant 51 includes the anchor portion 511 and a suture locking portion 512 connected with a proximal end of the anchor portion 511. The anchor portion 511 and the suture locking portion 512 are not separable in axial relative locations. The anchor portion 511 can be fixedly connected with the suture locking portion 512, or can be rotatably connected with the suture locking portion 512. The suture locking portion 512 is provided with a suture space 5120, and the suture space 5120 can axially penetrate through two ends of the suture locking portion 512. In the embodiment shown in FIG. 22, the suture 41 is always disposed in the suture space 5120 of the suture locking portion 512, so as to directly connect the suture 41 with the suture locking portion 512. In the embodiment shown in FIGS. 23 and 24, before the tension of the suture 41 is adjusted, the traction member 60 is always disposed in the suture space 5120 of the suture locking portion 512. Once the tension of the suture 41 starts to be adjusted, for example, the traction member 60 is tightened outside the body, the proximal end of the suture 41 located between the leaflet 211 and a distal end of the suture locking portion 512 can be pulled, and enter into the suture space 5120 from the distal end of the suture locking portion 512 to the proximal end of the suture locking portion 512 until out of the suture locking portion 512. Then the proximal end (i.e., the free end) of the suture 41 can be further adjusted slightly near the proximal end of the suture locking portion 512, until the suture 41 reaches the appropriate tension.

Once the suture 41 is adjusted to the appropriate tension, the ventricular implant 51 is actuated to retain the suture 41 under the appropriate tension. In some embodiments, the ventricular implant 51 is actuated by a locking actuator (not shown) to retain the suture 41 at the ventricular implant 51, such as to retain the suture 41 beside the proximal end of the suture locking portion 512. The locking actuator can actuate the ventricular implant 51 by utilizing, for example, a pressure force, a pulling force, a thrusting force, a rotation force, etc. For example, the suture locking portion 512 deforms or moves to lock the suture 41 in the ventricular implant 51.

In some embodiments, as shown in FIG. 25, the anchor portion 511 has a helical coil 511a, the suture locking portion 512 has a locking portion 512a with an elastic force, the helical coil 511a is rotatably connected to a distal end of the locking portion 512a, an anchor actuator 54 passes through a suture space 5120 of the locking portion 512a to be detachably connected with the helical coil 511a. The helical coil 511a can rotate relative to the locking portion 512a under helical advancing of the anchor actuator 54 loaded in the anchor catheter 52, so as to anchor a sharp distal end of the helical coil 511a into ventricular tissue. Once the suture 41 is adjusted to the appropriate tension, the locking portion 512a is actuated by the locking actuator loaded in the anchor catheter 52, so as to lock the suture 41 in the locking portion 512a at an appropriate location of the locking portion 512a.

An outer diameter, an effective length, a pitch and other parameters of the helical coil 511a can be reasonably designed according to actual acquirements, and a cross-sectional shape of the helical coil 511a can be, but not limited to, circular, quadrilateral, polygonal or irregular. The locking portion 512a completes the locking of the suture 41 by the pulling force. The locking portion 512a includes at least two connection plates 5121a and an elastic member 5122a axially disposed between the two connection plates 5121a. The elastic member 5122a includes a plurality of first helical sections 51221a and a plurality of second helical sections 51222a, at least one second helical section 51222a is disposed between each two adjacent first helical sections 51221a. The suture 41 or the traction member 60 penetrates the plurality of the first helical sections 51221a of the elastic member 5122a and extends to pass through the at least two connection plates 5121a. A proximal end of the helical coil 511a axially penetrates the distal connection plate 5121a and can rotate relative to the distal connection plate 5121a of the locking portion 512a, so as to guarantee that when the helical coil 511a is helically advanced to the ventricular tissue, the locking portion 512a cannot rotate along with the helical coil 511a to cause the problem of entangling with the suture 41 or the traction member 60. When the locking portion 512a is in an initial state in the anchor catheter 52, the proximal connection plate 5121a is tensioned and locked by the locking actuator connected with the connection plate 5121a, so as to make the elastic member 5122a continuously in a stretched state. When the locking actuator actuates the proximal connection plate 5121a to release the locking, the elastic member 5122a recovers to an original state under the action of an elastic restoring force, so as to clamp the suture 41 disposed in the locking portion 512a to complete the locking of the suture 41. Certainly, in other embodiments, the proximal end of the helical coil 511a can also be fixedly connected to a distal end of the distal connection plate 5121a.

In other embodiments, as shown in FIG. 26, the anchor portion 511 can further be an expandable implantation portion 511b which is radially expandable, and the expandable implantation portion 511b is fixedly connected to the distal end of the locking portion 512a. The expandable implantation portion 511b includes a main body 5111b and a plurality of inverted claws 5112b extending around the main body 5111b, the plurality of inverted claws 5112b can be bent outwards from the main body 5111b under the action of an elastic force to self-open or self-expand. Under axial advancing of the anchor actuator 54 loaded in the anchor catheter 52, the inverted claw 5112b is released from the opening at the distal end of the anchor catheter 52 and is automatically opened, so as to be anchored to ventricular tissue. In some embodiments, at least portion of a material of the inverted claw 5112b can be made of a shape memory material, such as a nickel-titanium alloy.

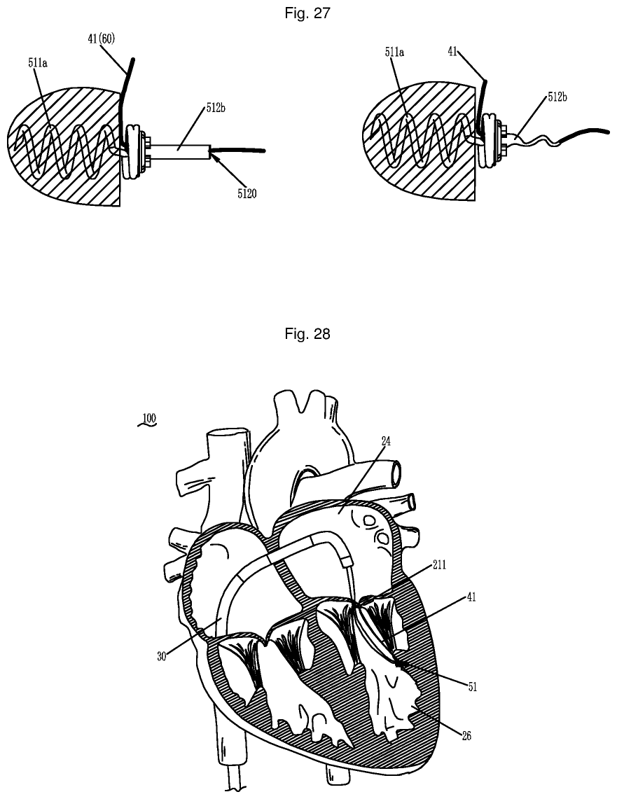

Certainly, in other embodiments, as shown in FIG. 27, the suture locking portion 512 can further be a locking pin 512b, and the locking pin 512b is fixedly connected to the proximal end of the helical coil 511a. The locking pin 512b has a hollow inner cavity penetrating two ends in an axial direction, so as to accommodate the suture 41 or the traction member 60. The locking pin 512b is a hollow thin-walled tube, and can be compressed when being subjected to an external mechanical force, so as to lock the suture 41 in the hollow inner cavity. A shape of the locking pin 512b is not limited to being cylindrical or prismatic, only if the locking pin 512b has a hollow inner cavity configured to accommodate the suture 41 or the traction member 60. Certainly, the hollow inner cavity of the locking pin 512b can further be in communication with a space surrounded by the plurality of helical sections of the helical coil 511a, and the suture 41 or the traction member 60 can simultaneously penetrate the hollow inner cavity and the space surrounded by the plurality of helical sections of the helical coil 511a. Then, when the helical coil 511a is actuated by the anchor actuator (not shown) loaded in the anchor catheter 52, so as to implement an anchoring operation, the suture 41 or the traction member 60 can be threaded out of the helical coil 511a to prevent winding. When the locking pin 512b is radially actuated by the locking actuator (not shown) loaded in the anchor catheter 52, the locking pin 512b can be compressed to lock the suture 41 in the hollow inner cavity.

The ventricular implantation subsystem 50 according to the present disclosure integrates an anchoring function and a suture locking function. That is, the anchoring function and the suture locking function involved in the present disclosure can be implemented by only one instrument. Therefore, different instruments do not need to be frequently replaced and introduced in a whole surgery process, thereby simplifying surgery steps, greatly shortening surgery duration, and further guaranteeing a surgery effect.

Once the anchoring of the ventricular implant 51 is completed, the anchor actuator is driven to disengage the anchor actuator from the ventricular implant 51, and the ventricular implantation subsystem 50 and the traction member 60 are withdrawn, or the ventricular implantation subsystem 50 is withdrawn.

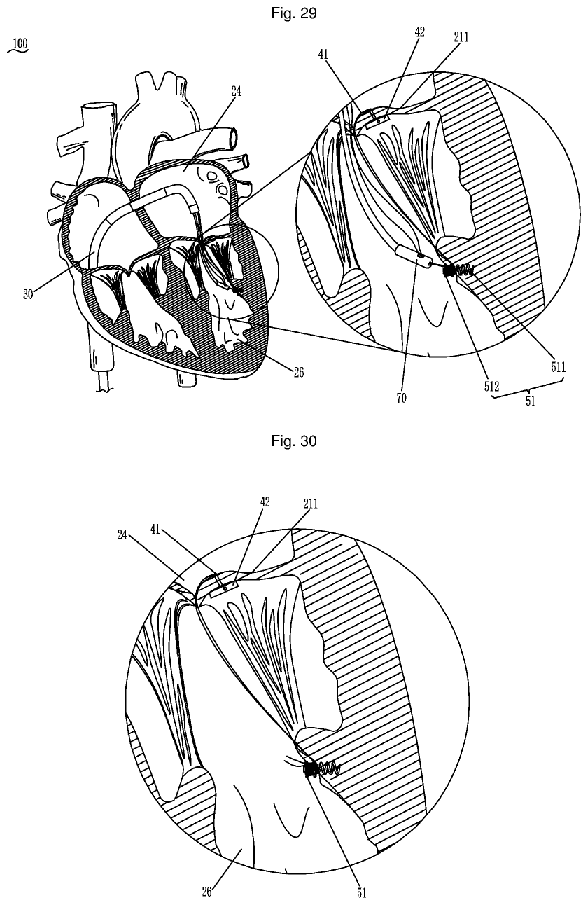

In some embodiments, as shown in FIG. 28, after the ventricular implant subsystem 50 is withdrawn, the suture 41 needs to be further cut near the ventricular implant 51, so as to leave the suture 41 between the leaflet implant 42 and the ventricular implant 51 as an artificial chordae tendineae. With reference to FIG. 29, the system for transcatheter chordae tendineae repair 100 can further include a suture cutter 70, and the suture 41 is connected to the suture cutter 70 outside the patient's body. The suture cutter 70 is advanced along the delivery catheter 30 to the heart near the ventricular implant 51, so as to cut the suture 41 near the ventricular implant 51 with a cutting member (not shown) in the suture cutter 70. For example, the suture 41 can be cut at the proximal end of the suture locking portion 512, so as to leave a remaining line segment of the suture 41 as the artificial chordae tendineae. After the heart valve has been repaired, the suture cutter 70 and the other line segment of the suture 41 can be withdrawn, and the delivery catheter 30 can be further withdrawn, so as to complete the implantation of the artificial chordae tendineae. A completion state of the implantation of the artificial chordae tendineae refers to FIG. 30.

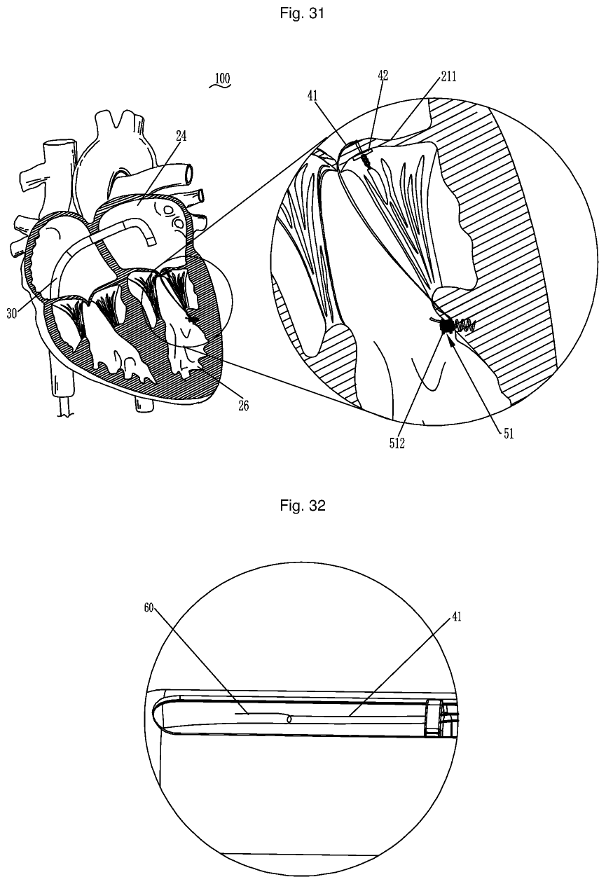

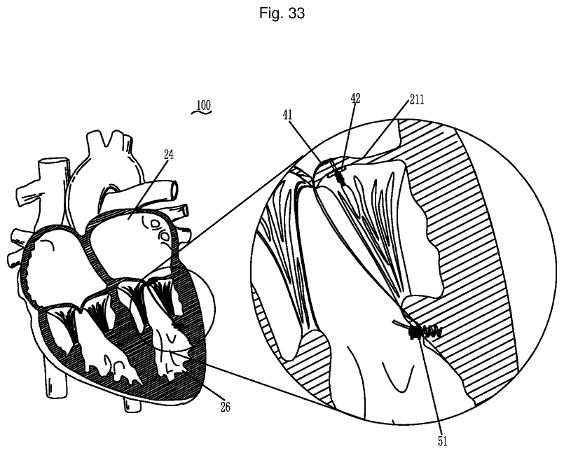

In other embodiments, as shown in FIG. 31, the ventricular implantation subsystem 50 and the traction member 60 are withdrawn, so as to leave the suture 41 as the artificial chordae tendineae. In some embodiments, the traction member 60 is firstly withdrawn. For example, one end of the traction member 60 passes out of the free end of the suture 41 in a manner as shown in FIG. 32 for withdrawing, so as to disengage connection between the traction member 60 and the suture 41. Then, the ventricular implantation subsystem 50 is withdrawn, and at this moment, the free end of the suture 41 is located near the ventricular implant 51. For example, the free end of the suture 41 is located at the proximal end of the suture locking portion 512. Then, the heart valve has been repaired. Therefore, the delivery catheter 30 can be further withdrawn to complete implantation of the artificial chordae tendineae. A completion state of the implantation of the artificial chordae tendineae refers to FIG. 33. The method and system for transcatheter chordae tendineae repair in the embodiments cut out a suture cutting step and the suture cutter. That is, implantation of the artificial chordae tendineae can be completed by only two sets of delivery systems for chordae tendineae repair, thereby greatly shortening surgery duration, and reducing the risk of thrombus caused by intervention instrument staying in a human body for a long time. Moreover, the embodiment further simplifies surgery steps, and reduces surgery complexity, thereby greatly improving a success rate of the surgery.

Certainly, for the embodiment shown in FIG. 31, if the artificial chordae tendineae reserved near the ventricular implant 51 (for example, reserved at the proximal end of the suture locking portion 512) is excessively long during the surgery, the suture cutter 70 can also be introduced to cut the artificial chordae tendineae which is excessively long, so as to guarantee that a length of the artificial chordae tendineae reserved near the ventricular implant 51 (i.e., the tail of the suture 41) is appropriate, so as to avoid the situation that the length of the tail of the suture 41 is excessively long to affect an endothelial crawling effect after the artificial chordae tendineae is implanted.

What are described above are merely the embodiments of the examples of the present invention. It should be pointed out that those of ordinary skill in the technical field can further make several improvements and modifications without departing from the principle of the examples of the present invention, and these improvements and modifications should also fall within the scope of protection of the present invention.

What is claimed is:

1. A method for transcatheter chordae tendineae repair, comprising:
transvascular entering into an atrium of a heart;
advancing a leaflet implant from an atrial side of a leaflet of the heart to a corresponding ventricular side, so as to fix the leaflet to a suture attached to the leaflet implant, wherein a proximal end of the suture extends out of a body to connect with a ventricular implant;
delivering the ventricular implant from the atrium to a corresponding ventricle, and anchoring the ventricular implant to ventricular tissue of the corresponding ventricle;
adjusting the suture to an appropriate tension; and
actuating the ventricular implant to retain the suture at the ventricular implant under the appropriate tension.

2. The method as claimed in claim 1, further comprising: cutting the suture near the ventricular implant, so as to leave the suture between the leaflet implant and the ventricular implant as an artificial chordae tendineae.

3. The method as claimed in claim 1, wherein a traction member is connected with the proximal end of the suture, and the traction member extends proximally out of the body to connect with the ventricular implant.

4. The method as claimed in claim 3, wherein a step of the adjusting the suture to an appropriate tension comprises: pulling proximally the traction member to bring at least partially of the proximal end of the suture to the ventricular implant for connecting.

5. The method as claimed in claim 4, wherein the ventricular implant comprises a suture locking portion, and a step of adjusting the suture to an appropriate tension further comprises: bringing the proximal end of the suture to pass through the suture locking portion from a distal end of the suture locking portion to a proximal end of the suture locking portion, so as to retain the proximal end of the suture beside the proximal end of the suture locking portion.

6. The method as claimed in claim 5, wherein a step of the actuating the ventricular implant comprises: actuating the suture locking portion to lock the suture in the suture locking portion.

7. The method as claimed in claim 3, further comprising: connecting the traction member to the proximal end of the suture outside the body.

8. The method as claimed in claim 3, further comprising: withdrawing the traction member, so as to leave the suture as an artificial chordae tendineae.

9. The method as claimed in claim 3, further comprising: cutting the suture at the proximal end of the suture, and withdrawing the traction member.

10. The method as claimed in claim 1, wherein a step of the anchoring the ventricular implant to ventricular tissue comprises: rotating the ventricular implant into the ventricular tissue.

11. The method as claimed in claim 10, wherein a step of the anchoring the ventricular implant to ventricular tissue further comprises: inserting a locator into the ventricular tissue before rotating the ventricular implant.

12. A system for transcatheter chordae tendineae repair, comprising:
a delivery catheter, wherein the delivery catheter is advanced into an atrium of a heart through a vasculature of a patient, so as to provide a pathway into the heart from outside a body;
a leaflet implant to which a suture is attached, wherein the leaflet implant is configured to be advanced from an atrial side of a leaflet to a corresponding ventricular side through the delivery catheter, so as to fix the leaflet to the suture; and
a ventricular implant, wherein the suture extends proximally through the delivery catheter to be connected with the ventricular implant outside the body, and the ventricular implant is configured to be advanced from the atrium to a corresponding ventricle through the delivery catheter, so as to be anchored to ventricular tissue;
when the suture being adjusted to an appropriate tension, actuating the ventricular implant to retain the suture at the ventricular implant.

13. The system as claimed in claim 12, further comprising: a puncture assembly, wherein the leaflet implant is preloaded in the puncture assembly, the suture extends proximally through the puncture assembly, and the puncture assembly is delivered into the atrium through the delivery catheter to puncture the leaflet, so as to release the leaflet implant to the ventricular side from the puncture assembly.

14. The system as claimed in claim 13, further comprising: a capture assembly, wherein the capture assembly is delivered via the delivery catheter, until a distal end of the capture assembly is beyond a distal end of the delivery catheter.

15. The system as claimed in claim 14, wherein the puncture assembly is loaded in the capture assembly.

16. The system as claimed in claim 12, wherein the ventricular implant comprises a suture locking portion, and a proximal end of the suture is brought to pass through the suture locking portion from a distal end of the suture locking portion to a proximal end of the suture locking portion, so as to retain the proximal end of the suture beside the proximal end of the suture locking portion.

17. The system as claimed in claim 16, wherein the suture locking portion is capable of being actuated to lock the suture in the suture locking portion.

18. The system as claimed in claim 17, further comprising: a locking actuator, wherein the locking actuator is pushed along the suture and is delivered to the ventricle via the delivery catheter, so as to actuate the suture locking portion to lock the suture.

19. The system as claimed in claim 12, further comprising: a suture cutter, wherein the suture cutter is configured to cut the suture near the ventricular implant, so as to leave the suture between the leaflet implant and the ventricular implant as an artificial chordae tendineae.

20. The system as claimed in claim 12, further comprising: a traction member, wherein a proximal end of the suture is connected with the traction member, and the traction member extends proximally out of the body through the delivery catheter to connect with the ventricular implant outside of the body.

21. The system as claimed in claim 20, wherein the traction member pulls the suture, so as to bring at least partially of the proximal end of the suture to the ventricular implant for connecting.

22. The system as claimed in claim 21, wherein the suture is being left as an artificial chordae tendineae after the traction member is withdrawn.

23. The system as claimed in claim 22, further comprising: a suture cutter, wherein the suture cutter is configured to cut the suture at the proximal end of the suture, so as to leave the suture between the leaflet implant and the ventricular implant as the artificial chordae tendineae.

24. The system as claimed in claim 12, further comprising: a locator, wherein the locator is configured to be inserted into the ventricular tissue before the ventricular implant is anchored through the delivery catheter.
